(19) United States
(12) Patent Application Publication
(10) Pub. No.: US 2008/0176227 A1
(43) Pub. Date: Jul. 24, 2008

(54) Dna Carrier, Method of Producing the Same and Collection System Using the Same

(75) Inventors: Zuyi Zhang, Kanagawa-ken (JP); Teigo Sakakibara, Tokyo (JP); Yoshinori Kotani, Kanagawa-ken (JP); Kazuyuki Matsumura, Gunma-ken (JP); Norio Nishi, Hokkaido (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/632,174

(22) PCT Filed: Jul. 13, 2005
(86) PCT No.: PCT/JP2005/013344

§ 371 Date: Sep. 7, 2007

(30) Foreign Application Priority Data
Jul. 14, 2004 (JP) 2004-207253

(51) Int. Cl. C12Q 1/68

(52) U.S. Cl. 435/6

(57) ABSTRACT

Provided are a DNA carrier where DNA is firmly held in a substrate, which can reduce the elution of DNA into water and can take full advantage of the capability of DNA to selectively and specifically collect a substance; and a method of producing the DNA carrier. Also provided is a collection system using DNA, which can be used in the high-accuracy detection of a particular substance and in an environmental cleanup capable of efficiently removing a substance, in which the DNA carrier is used to collect the substance contained in air or water by taking full advantage of the capability of DNA to selectively and specifically collect the substance. The DNA carrier is one where DNA is held in a porous matrix containing polyorganosiloxane with a basic functional group and particles. Preferably, the polyorganosiloxane with a basic functional group contains a hydrolysis condensate of one or more of particular silane compounds with an amino group.

### TECHNICAL FIELD

The present invention relates to a DNA carrier. In particular, the present invention relates to a DNA carrier where DNA is firmly held in a porous matrix, which reduces the elution of DNA into water even when brought into contact with water and retains the selective recognition capability of DNA and the capability of incorporating a particular substance into the double helix of DNA; a method of producing the DNA carrier; and a collection system using DNA.

### BACKGROUND ART

DNA (deoxyribonucleic acid) is responsible for genetic information in living bodies and is one of the most important substances for vital phenomena. DNA has the extremely accurate ability to recognize molecules because single strand DNA together with complementary single strand DNA forms a number of base pairs. DNA undergoes the selective and specific intercalation (incorporation), into its double helix, of aromatic compounds having planer chemical structures and as such, is regarded as a potential material for detecting carcinogenic compounds present in water or air and for environmental cleanup to remove harmful substances (Kino Zairyo in Japanese (Functional Materials), Vol. 19, 1999). The application of such DNA to a material for detection and environmental cleanup requires the immobilization of water-soluble DNA on a substrate, so that the development of techniques for immobilizing DNA on a substrate is pushed forward. There have been reported, for example, a method having the step of bringing the surface of a support into contact with DNA in a buffer solution containing morpholine, a morpholine derivative and/or a salt thereof (International Patent Publication No. WO 00/34456); a method of immobilizing DNA through a polymer modified with γ-aminopropyltriethoxysilane (Chem. Rev., Vol. 96, 1533-1554, 1996; Anal. Chim. Acta, Vol. 365, 19-25, 1998); a method for nucleic acid immobilization in which a substrate is treated with atomic oxygen plasma (Japanese Patent Application Laid-Open No. 2002-218976); a method of immobilizing a deoxyribonucleic acid by using a divalent metal-containing compound to coagulate an alkaline metal salt of the deoxyribonucleic acid and an alkaline metal salt of alginic acid (see e.g., Japanese Patent Application Laid-Open No. H07-41494); a method in which DNA is solidified and immobilized on a support by irradiating the aqueous solution or thin solution film of water-soluble DNA on the support or the thin film of water-soluble DNA on the support with ultraviolet light having wavelengths in the range of 250 to 270 nm (see e.g., Japanese Patent Application Laid-Open No. 2001-81098); and a DNA-immobilized composite material containing a calcium-containing substance or inorganic solid material such as silica gel as a DNA-immobilized carrier (see e.g., Japanese Patent Application Laid-Open No. H10-175994).

These methods render DNA water-insoluble by holding DNA on a substrate or by bringing about the cross-linking reaction of DNA. However, in these methods, there remain problems that the exposed area of DNA is small and the capability of DNA is not fully exploited.

### DISCLOSURE OF THE INVENTION

The present invention provides a DNA carrier where DNA is firmly held in a substrate, which can reduce the elution of DNA into water and can take full advantage of the capability of DNA to selectively and specifically collect a substance; and a method of producing the DNA carrier. The present invention further provides a collection system using DNA, which can be used in the high-accuracy detection of a particular substance and in an environmental cleanup capable of efficiently removing a substance, in which the DNA carrier is used to collect the substance contained in air or water by taking full advantage of the capability of DNA to selectively and specifically collect the substance.

The present inventors have already developed a DNA hybrid where DNA is held in a porous oxide matrix by removing a dispersion solvent from a dispersion solution containing a colloidal oxide and the DNA. The present inventors have further diligently studied and consequently completed the present invention by finding out that DNA is held in a porous matrix containing polyorganosiloxane with a basic functional group and particles to thereby allow significant reduction in the elution of DNA into water.

That is, the present invention includes the following aspect:

a DNA carrier, characterized in that DNA is held in a porous matrix containing polyorganosiloxane with a basic functional group and particles.

The DNA carrier of the present invention where DNA is firmly held in a substrate can reduce the elution of DNA into water and can take full advantage of the capability of DNA to selectively and specifically collect a substance. The method of producing the DNA carrier of the present invention can be used to conveniently produce such a DNA carrier. The collection system using DNA of the present invention can be applied to the high-accuracy detection of a particular substance and in an environmental cleanup system capable of efficiently removing a particular substance, in which DNA collects a particular substance contained in air or water by taking full advantage of the capability of the DNA to selectively and specifically collect the substance.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a diagrammatic view showing a collection system of the present invention.

### BEST MODE FOR CARRYING OUT THE INVENTION

Preferred embodiments of the present invention will now be described in detail in accordance with the accompanying drawings.

A DNA carrier of the present invention is not particularly limited as long as it is a DNA carrier where DNA is held in a porous matrix containing polyorganosiloxane with a basic functional group and particles.

The polyorganosiloxane with a basic functional group contained in the porous matrix in the DNA carrier of the present invention combines the capability to form a matrix with the capability to hold DNA. Such capability to hold DNA is derived from the basic functional group in the polyorganosiloxane, which forms an acid-base structure with a phosphate group of DNA to thereby allow DNA to be firmly held in the porous matrix, with its double helix maintained. The polyorganosiloxane with a basic functional group is preferably any of those facilitating the preparation of an uniform dispersion/dissolution solution with particles (which will be described below) contained in the porous matrix and with DNA when the DNA carrier is produced. Preferred polyorganosiloxane is a water-soluble hydrolysis condensate obtained by hydrolyzing a silane compound with a basic functional group.

The silane compound with a basic functional group that can form such polyorganosiloxane with a basic functional group is a silane compound that has a basic functional group having the capability to hold DNA in polyorganosiloxane as well as a hydrolyzable functional group, and may also be a silane compound that has an alkyl substituent. The hydrolyzable functional group can include a halogen atom and an alkoxy group, with the alkoxy group preferred. Examples of the alkoxy group can include an alkoxy group having 1 to 8 carbon atoms such as methoxy, ethoxy, n-propoxy and n-butoxy groups. Examples of the alkyl group used as a substituent can include an alkyl group having 1 to 8 carbon atoms such as methyl, ethyl, n-propyl and n-butyl groups. The basic functional group of the silane compound is the same as that of the polyorganosiloxane described above. Such a basic functional group is preferably an amino group and may also be a primary amino group, with secondary, tertiary and quaternary amino groups particularly preferred. Concrete examples thereof can include an alkylamino group having 1 to 8 carbon atoms such as methylamino, dimethylamino and ethylamino groups and an N-containing heterocyclic group. Preferred concrete examples of such a silane compound can include any one or more of compounds represented by the formula (1):

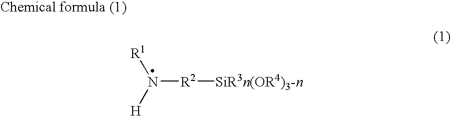

wherein R² represents a divalent carbohydrate group having 1 to 8 carbon atoms or a divalent group having —NH—; when R² represents a divalent carbohydrate group having 1 to 8 carbon atoms, R¹represents a monovalent carbohydrate group having 1 to 8 carbon atoms, and when R² represents a divalent group having —NH—, R¹ represents H or a monovalent carbohydrate group having 1 to 8 carbon atoms; R³ and R⁴ each independently represent a monovalent carbohydrate group having 1 to 8 carbon atoms; and n represents 0, 1 or 2;

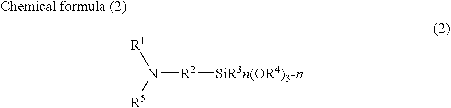

wherein R¹, R³, R⁴ and R⁵ each independently represent a monovalent carbohydrate group having 1 to 8 carbon atoms; R² represents a divalent carbohydrate group having 1 to 8 carbon atoms or a divalent group having —NH—; and n represents 0, 1 or 2;

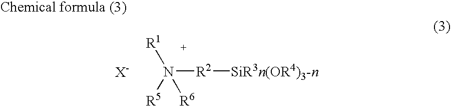

wherein R¹, R³, R⁴, R⁵ and R⁶ each independently represent a monovalent carbohydrate group having 1 to 8 carbon atoms; R² represents a divalent carbohydrate group having 1 to 8 carbon atoms or a divalent group having —NH—; n represents 0, 1 or 2; and X⁻ represents an anion;

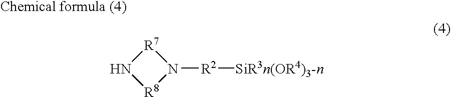

wherein R³ and R⁴ each independently represent a monovalent carbohydrate group having 1 to 8 carbon atoms; R⁷ and R⁸ each independently represent a divalent carbohydrate group; R² represents a divalent carbohydrate group having 1 to 8 carbon atoms or a divalent group having —NH—; and n represents 0, 1 or 2;

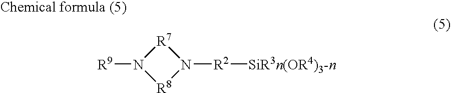

wherein R³, R⁴ and R⁹ each independently represent a monovalent carbohydrate group having 1 to 8 carbon atoms; R⁷ and R⁸ each independently represent a divalent carbohydrate group; R² represents a divalent carbohydrate group having 1 to 8 carbon atoms or a divalent group having —NH—; and n represents 0, 1 or 2. Examples of the monovalent carbohydrate group having 1 to 8 carbon atoms represented by R¹, R³, R⁴, R⁵, R⁶ or R⁹ in the formulas (1) to (5) can include a chain, branched or cyclic alkyl group having 1 to 8 carbon atoms such as methyl, ethyl, n-propyl, s-propyl, n-butyl, s-butyl, t-butyl, n-pentyl, n-hexyl, n-heptyl and n-octyl groups and an aromatic carbohydrate group such as a phenyl group. The divalent carbohydrate group having 1 to 8 carbon atoms represented by R² in the formulas (1) to (5) can include a chain, branched or cyclic divalent alkylene group having 1 to 8 carbon atoms such as methylene, ethylene, trimethylene and tetramethylene groups and a divalent aromatic carbohydrate group having 1 to 8 carbon atoms such as o-phenylene, m-phenylene and p-phenylene groups. The divalent group having —NH— can concretely include —NH and a group formed by the bonding of one or two of divalent carbohydrate groups such as methylene, ethylene, trimethylene and tetramethylene groups to a nitrogen atom, which can concretely exemplified by —C₂H₄NHC₃H₆—, —C₃H₆NHC₂H₄—, —CH₂NHC₃H₆—, —C₂H₄NHCH₂—, —C₂H₄NHC₂H₄— and —C₃H₆NHC₃H₆—. The divalent carbohydrate group represented by R⁷ or R⁸ in the formulas (4) to (5) is not limited by the number of a carbon atom and can include a chain, branched or cyclic divalent alkylene group such as methylene, ethylene, trimethylene and tetramethylene groups and a divalent aromatic carbohydrate group such as o-phenylene, m-phenylene and p-phenylene groups. It can concretely be exemplified by methylene and ethylene groups. The anion represented by X⁻ in the formula (3) may be any of those capable of forming an ion pair with the cation of siloxane having a quaternary amino group and can include a halogen ion.

The compounds represented by the above-described formulas (1) to (3) can concretely include (CH₃)HNC₃H₆Si(OCH₃)₃, (CH₃)HNC₃H₆SiCH₃(OCH₃)₂, (CH₃)HNC₃H₆Si(OC₂H₅)₃, (CH₃)HNC₃H₆SiCH₃(OC₂H₅)₂, (CH₃)₂NC₃H₆Si(OCH₃)₃, (CH₃)₂NC₃H₆SiCH₃(OCH₃)₂, (CH₃)₂NC₃H₆Si(OC₂H₅)₃, (CH₃)₂NC₃H₆SiCH₃(OC₂H₅)₂, (C₂H₅)₂NC₃H₆Si(OCH₃)₃, (C₂H₅)₂NC₃H₆Si(OC₂H₅)₃, H₂NC₂H₄NHC₃H₆Si(OCH₃)₃, H₂NC₂H₄NHC₃H₆SiCH₃(OCH₃)₂, H₂NC₂H₄NHC₃H₆Si(OC₂H₅)₃, H₂NC₂H₄NHC₃H₆SiCH₃(OC₂H₅)₂, (CH₃)HNC₂H₄NHC₃H₆Si(OCH₃)₃, (CH₃)HNC₂H₄NHC₃H₆SiCH₃(OCH₃)₂, (CH₃)HNC₂H₄NHC₃H₆Si(OC₂H₅)₃, (CH₃)HNC₂H₄NHC₃H₆SiCH₃(OC₂H₅)₂, (CH₃)₂NC₂H₄NHC₃H₆Si(OCH₃)₃, (CH₃)₂NC₂H₄NHC₃H₆SiCH₃(OCH₃)₂, (CH₃)₂NC₂H₄NHC₃H₆Si(OC₂H₅)₃, (CH₃)₂NC₂H₄NHC₃H₆SiCH₃(OC₂H₅)₂, Cl⁻(CH₃)₃N⁺C₃H₆Si(OCH₃)₃, Cl⁻(C₄H₉)₃N⁺C₃H₆Si(OCH₃)₃ shown in Table 1.

TABLE 1

| | For- | | | | | | | | |
|---|---|---|---|---|---|---|---|---|---|
| | mula | R¹ | R⁵ | R⁶ | R² | R³ | R⁴ | n | X⁻ |
| 1 | 1 | CH₃ | — | — | C₃H₆ | — | CH₃ | 0 | — |
| 2 | 1 | CH₃ | — | — | C₃H₆ | CH₃ | CH₃ | 1 | — |
| 3 | 1 | CH₃ | — | — | C₃H₆ | — | C₂H₅ | 0 | — |
| 4 | 1 | CH₃ | — | — | C₃H₆ | CH₃ | C₂H₅ | 1 | — |
| 5 | 2 | CH₃ | CH₃ | — | C₃H₆ | — | CH₃ | 0 | — |
| 6 | 2 | CH₃ | CH₃ | — | C₃H₆ | CH₃ | CH₃ | 1 | — |
| 7 | 2 | CH₃ | CH₃ | — | C₃H₆ | — | C₂H₅ | 0 | — |
| 8 | 2 | CH₃ | CH₃ | — | C₃H₆ | CH₃ | C₂H₅ | 1 | — |
| 9 | 2 | C₂H₅ | C₂H₅ | — | C₃H₆ | — | CH₃ | 0 | — |
| 10 | 2 | C₂H₅ | C₂H₅ | — | C₃H₆ | — | C₂H₅ | 0 | — |
| 11 | 1 | H | — | — | C₂H₄NHC₃H₆ | — | CH₃ | 0 | — |
| 12 | 1 | H | — | — | C₂H₄NHC₃H₆ | CH₃ | CH₃ | 1 | — |
| 13 | 1 | H | — | — | C₂H₄NHC₃H₆ | — | C₂H₅ | 0 | — |
| 14 | 1 | H | — | — | C₂H₄NHC₃H₆ | CH₃ | C₂H₅ | 1 | — |
| 15 | 1 | CH₃ | — | — | C₂H₄NHC₃H₆ | — | CH₃ | 0 | — |
| 16 | 1 | CH₃ | — | — | C₂H₄NHC₃H₆ | CH₃ | CH₃ | 1 | — |
| 17 | 1 | CH₃ | — | — | C₂H₄NHC₃H₆ | — | C₂H₅ | 0 | — |
| 18 | 1 | CH₃ | — | — | C₂H₄NHC₃H₆ | CH₃ | C₂H₅ | 1 | — |
| 19 | 2 | CH₃ | CH₃ | — | C₂H₄NHC₃H₆ | — | CH₃ | 0 | — |
| 20 | 2 | CH₃ | CH₃ | — | C₂H₄NHC₃H₆ | CH₃ | CH₃ | 1 | — |
| 21 | 2 | CH₃ | CH₃ | — | C₂H₄NHC₃H₆ | — | C₂H₅ | 0 | — |
| 22 | 2 | CH₃ | CH₃ | — | C₂H₄NHC₃H₆ | CH₃ | C₂H₅ | 1 | — |
| 23 | 3 | CH₃ | CH₃ | CH₃ | C₃H₆ | — | CH₃ | 0 | Cl⁻ |
| 24 | 3 | C₄H₉ | C₄H₉ | C₄H₉ | C₃H₆ | — | CH₃ | 0 | Cl⁻ |

The compounds represented by the above-described formulas (4) and (5) can concretely include compounds represented by the formulas (4) and (5) in which R², R⁷ and R⁸ each represent, for example, a divalent carbohydrate group such as methylene, ethylene and trimethylene groups and R³, R⁴ and R⁹ each represent a monovalent carbohydrate group such as methyl, ethyl and propyl groups. Preferred examples thereof can include a compound represented by the formula (6):

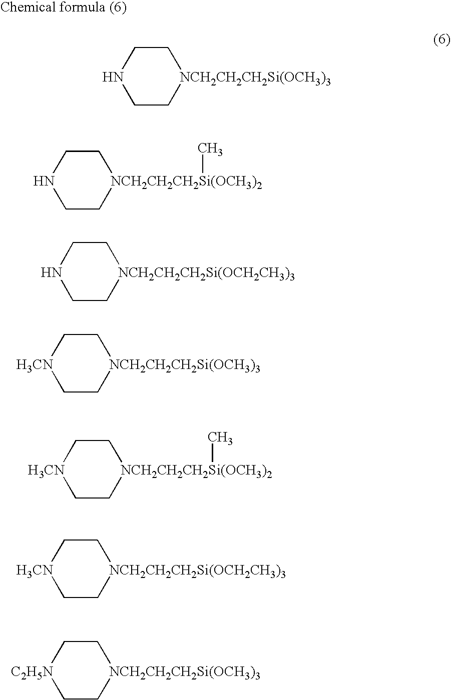

The polyorganosiloxane with a basic functional group applied to the present invention is a siloxane compound with a basic functional group, preferably a water-soluble hydrolysis condensate with a basic functional group that can be obtained by hydrolyzing any one or more of the silane compounds with a basic functional group represented by the above-described formulas (1) to (5), and may optionally be any of those containing an alkylsiloxane component or a phenylsiloxane component. As an example, the polyorganosiloxane with a basic functional group that contains such a component may be a copolymer obtained by adding, for example, an alkylalkoxysilane compound or a phenylalkoxysilane compound to the above-described silane compound with a basic functional group, which is in turn subjected to hydrolysis and condensation polymerization. The alkylalkoxysilane can include CH₃Si(OCH₃)₃, CH₃Si(OC₂H₅)₃, (CH₃)₂Si(OCH₃)₂ and (CH₃)₂Si(OC₂H₅)₂. The phenylalkoxysilane can include C₆H₅Si(OCH₃)₃ and C₆H₅Si(OC₂H₅)₃.

In procedures for hydrolyzing a silane compound with a basic functional group to form polyorganosiloxane with a basic functional group, the silane compound with a basic functional group may directly be added to water and then hydrolyzed; or otherwise the silane compound with a basic functional group may also be hydrolyzed after being dissolved in an alcohol, ketone or the like and then added to water or after being added to the mixed dispersion solvent of an organic dispersion solvent such as alcohol or ketone with water. Those containing an organic dispersion solvent may be subjected to solvent replacement by water, as necessary, to obtain an aqueous dispersion solution of siloxane with a basic functional group.

The particles contained in the porous matrix in the DNA carrier of the present invention are components that form a number of pores in a matrix holding DNA therein to make the matrix porous. The pores formed in the matrix have the capability to hold DNA and the capability to promote the contact of DNA with a substance to be captured by the DNA. The particles forming such pores each have a particle size of preferably 5 to 100 nm, more preferably 10 to 50 nm. If the particle size of the particle is 5 nm or more, the size of the pore is large and DNA undergoes sufficient contact with a substance to be captured by the DNA. The particle having a particle size of 10 nm or more produces such an effect more remarkably. On the other hand, if the particle size of the particle is 100 nm or less, the pore can be secured in large numbers while the elution of DNA into an aqueous solution is reduced and the DNA is therefore firmly held in a porous matrix. The particle having a particle size of 50 nm or less produces such an effect more remarkably. It is noted that the value of the particle size of the particle used herein is measured by a laser diffraction method, a dynamic scattering method or the like.

It is preferred that the particle having a size capable of forming such a pore should be composed of a water-insoluble material. Examples of the material for the particle can include a plastic, a metal halogen compound and an oxide, with the oxide particularly preferred in light of an affinity for the above-described polyorganosiloxane with a basic functional group and the ease of availability. The oxide used as a material for such a particle can concretely include silicon dioxide, aluminum oxide, iron oxide, gallium oxide, lanthanum oxide, titanium oxide, cerium oxide, zirconium oxide, tin oxide and hafnium oxide. These oxides may be used alone or in combination of two or more. Particles of these oxides that become colloidal in an aqueous dispersion/dissolution solution are preferred because they are easy to uniformly mix in a dispersion/dissolution solution of the polyorganosiloxane with a basic functional group and facilitate the formation of the porous matrix. The colloidal oxide can concretely include colloidal particles of the oxides illustrated above. Of these colloidal particles, colloidal silicon dioxide is particularly preferred in light of an affinity for the polyorganosiloxane with a basic functional group and cost efficiency. A commercially-available product can be applied to such colloidal silicon dioxide. The commercially-available colloidal silicon dioxide that can be used is, for example, an aqueous sol such as SNOWTEX 20, SNOWTEX30, SNOWTEX N, SNOWTEX O and SNOWTEX C (trade names, manufactured by NISSAN CHEMICAL INDUSTRIES, LTD.), a solvent-based sol such as IPA-ST, EG-ST and MEK-ST (trade names, manufactured by NISSAN CHEMICAL INDUSTRIES, LTD.), and a solvent-based sol such as OSCAL-1132, OSCAL-1432 and OSCAL-1232 (trade names, manufactured by CATALYSTS&CHEMICALS IND. CO., LTD). Alternatively, for example, ALUMINASOL 100 and ALUMINASOL 520 (trade names, manufactured by NISSAN CHEMICAL INDUSTRIES, LTD.) can be used as colloidal aluminum oxide.

In the DNA carrier of the present invention, the porous matrix contains the polyorganosiloxane with a basic functional group and the particles in the ratio of the polyorganosiloxane with a basic functional group/particles ranging preferably from 0.1/99.9 to 25/75 by weight, more preferably from 0.5/99.5 to 10/90. If the ratio of the polyorganosiloxane with a basic functional group/particles is 0.1/99.9 or more by weight, DNA is appropriately held through the bonding between the phosphate group of the DNA. The ratio of 0.5/99.5 or more by weight produces such an effect more remarkably. On the other hand, if the ratio of the polyorganosiloxane with a basic functional group/particles is 25/75 or less by weight, pores are efficiently formed in the gaps among the particles. The ratio of 10/90 or less by weight produces such an effect more remarkably.

The porous matrix of the present invention may contain, for example, other components such as a surfactant within the bounds of not impairing the capability of DNA, in addition to the above-described polyorganosiloxane with a basic functional group and particles. As used herein, the term “porous” or “porosity” means that a liquid medium such as water infiltrates in the DNA carrier to result in increase in apparent density when the DNA carrier is immersed in the liquid medium. The degree of porosity of the porous matrix in the DNA carrier of the present invention is preferably 0.5% or more, more preferably 5% or more, in terms of increase in apparent density or weight when the porous matrix having DNA held therein reaches sufficient equilibrium in a solution.

The DNA used in the DNA carrier of the present invention is not limited by type and size as long as it can attain the object of the present invention, with it held in the porous matrix. That is, the DNA may be any of single strand DNA and double strand DNA, and no particular limitation is imposed on its molecular weight. The DNA that can be used includes DNA that may be cDNA, RNA that may be mRNA, a nucleic acid such as oligonucleotide and polynucleotide from a precursor of DNA. Such DNA that can be used is exemplified by DNA obtained from a testis or the thymus from an animal and to be more specific, DNA obtained from a soft roe (testis) from a salmon, herring or cod and DNA obtained from the thymus from a mammal or birds (e.g., a cow, a pig and a chicken). Synthetic DNA having a DNA sequence with (dA)-(dT) base pairs, specifically a poly(dA•dT)-poly(dA•dT) type sequence, can also be used. The double strand DNA is particularly preferred because it is enhanced in the effect of collecting a particular substance (i.e., the intercalation of a particular substance into DNA). The molecular weight of such DNA can be preferably 100,000 or higher, more preferably 500,000 or higher. If the molecular weight of the DNA is 100,000 or higher, the DNA can be immobilized with high efficiency in the matrix composed of the polyorganosiloxane with a basic functional group and the particles. The DNA having a molecular weight of 500,000 or higher produces such an effect more remarkably.

The DNA carrier of the present invention has a DNA content of preferably 0.01 to 15% (w/w), more preferably 0.1 to 10% (w/w). If the DNA content is 0.01% (w/w) or more, the DNA carrier can sufficiently attain the effect of collecting a particular substance by DNA. The DNA carrier having a DNA content of 0.1% (w/w) or more can produce such an effect more remarkably. On the other hand, if the DNA content is 15% (w/w) or less, no blockage occurs in pores in the porous matrix, resulting in an advantage from an economical point of view. The DNA carrier having a DNA content of 10% (w/w) or less can produce such an effect more remarkably. This accelerates the flow rate of gas or an aqueous solution coming into or out of the DNA carrier and allows DNA in the surface layer or in the pores of the porous matrix to exhibit the capability to collect a particular substance sufficiently and efficiently.

A method of producing the DNA carrier of the present invention can include a method in which DNA is held in a porous matrix simultaneously with the formation of the porous matrix by the steps of preparing a dispersion/dissolution solution where the above-described particles, DNA and polyorganosiloxane with a basic functional group are dispersed and dissolved; and removing a dispersion solvent from the dispersion/dissolution solution.

As used in the present invention, the dispersion/dissolution solution refers to a solution containing a substance in a state of dispersion, a solution containing a substance in a state of dissolution or both. The step of preparing a dispersion/dissolution solution can include a procedure in which a dispersion/dissolution solution of each of the above-described components is prepared and mixed together. A dispersion solution of the above-described particles can be prepared by using, for example, a commercially-available aqueous sol of particles or a solvent-based sol such as methanol and adjusting its concentration. A dispersion solution of the above-described polyorganosiloxane with a basic functional group can be prepared by adding, for example, a silane compound with a basic functional group dropwise to water to generate an oligomer with stirring, for example, at room temperature for 1 to 5 days, which is in turn concentrated at approximately 10 to 80° C., followed by the adjustment of the concentration of the solid content. Alternatively, a silane compound with a basic functional group may directly be hydrolyzed in a dispersion solution of particles. A dispersion/dissolution solution of the above-described DNA can be prepared by dispersing and dissolving, for example, natural DNA extracted from an animal organ in ion-exchanged water, for example, at 5° C. over 10 hours to 5 days and adjusting its concentration. The step of removing a dispersion solvent can include a procedure in which a dispersion solvent is removed from a dispersion/dissolution solution containing-particles, DNA, polyorganosiloxane with a basic functional group by a certain method such as heating, spray draying and vacuum drying. Such a method for removing a dispersion solvent can appropriately be selected according to the desired form of the DNA carrier, for example, a powder or a bulk. When the DNA carrier is used in the form of a powder, a dispersion/dissolution solution can be changed into a powder by spray drying. Alternatively the DNA carrier in the form of a powder can be obtained by forming a bulk and then pulverizing the obtained bulk. When the DNA carrier is formed into a film, such a powder is used to prepare a coating solution, which can then be used as a coating film that is applied to the surface of a substrate such as a plate, a tubular material, a fiber, a woven fabric and a nonwoven fabric. It is preferred that heat should be imparted to the resultant DNA carrier within the bounds not bringing about the decomposition of the DNA, after the step of removing a dispersion/dissolution solution as above. A temperature at which the porous DNA carrier is, heat-treated is preferably 200° C. or lower, more preferably 150° C. or lower.

The form of the DNA carrier of the present invention can include a powder, a bulk and a coating film that is applied to the surface of a substrate such as a plate, a tubular material, a fiber, a woven fabric and a nonwoven fabric as well as a module composed of the DNA carrier in any of these forms, for example, a column packed with the powder.

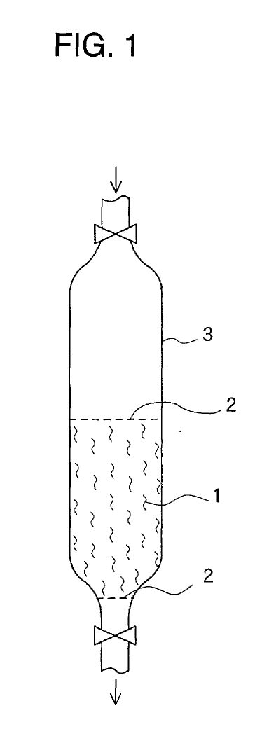

A collection system using DNA of the present invention is not particularly limited as long as it has means for bringing water and/or gas containing a substance that can be collected by DNA into contact with the DNA carrier of the present invention. Such means can include a module composed of a powder, a bulk and a coating film that is applied to the surface of a substrate such as a plate, a tubular material, fiber, a woven fabric and a nonwoven fabric, which are used as the DNA carrier of the present invention. Concrete examples thereof can include a column 3 in which a DNA carrier 1 in the form of fiber or the like is packed into a filter 2 as shown in FIG. 1, and a filter medium and an adsorbing member in which the material, shape and so on of a substrate that forms a coating film are appropriately selected.

Such a collection system can include cigarette filter, a filter medium for beverages and milk, an adsorbing/cleaning member used in the digestive canal or the like of a mammal including a human, and an environmental cleanup system for removing a harmful substance from air, drain and waste water from various sites, and water such as rivers, lakes and mashes. The environmental cleanup system can be exemplified by a system in which air or water containing a harmful substance is passed into a column packed with a powder or the like of the DNA carrier to thereby clean the harmful substance.

The harmful substance used herein refers to a compound that jeopardizes the structure or genetic information of DNA by interacting with the DNA through intercalation or adsorption. Substances that can interact with DNA have not been elucidated in part and however, can include harmful substances having an aromatic functional group that causes intercalation into DNA and heavy metal ions that is selectively adsorbed by DNA. Specific examples thereof can include dioxins such as polychlorodibenzo-para-dioxin, polychlorodibenzofurane and polychlorobiphenyl (PCB), benzo[a]pyrene, dichlorodiphenyltrichloroethane (DDT), diethylstilbestrol (DES), ethidium bromide, acridine orange and derivatives thereof.

Moreover, the collection system of the present invention can be applied to a detection system for a substance that can be collected by the DNA in the DNA carrier of the present invention. For example, a module of the DNA carrier of the present invention can be applied to the detection of a particular substance in the blood vessel or the digestive canal.

### EXAMPLES

### Synthesis Example 1

Forty grams of N,N-dimethylaminopropyltrimethoxysilane (207.34→138.34) was added dropwise to 200 g of distilled water and hydrolyzed at room temperature for 3 days. The resultant oligomer solution was concentrated at 60° C. with an evaporator. Thereafter, 80 g of distilled water was added thereto to obtain approximately 180 g of a siloxane solution with a basic functional group (N1) whose solid content was 14.8%.

### Synthesis Example 2

Forty grams of N-methylaminopropyltrimethoxysilane (193.32→124.32) was added dropwise to 200 g of distilled water and hydrolyzed at room temperature for 3 days. The resultant oligomer solution was concentrated at 60° C. with an evaporator. Thereafter, 70 g of distilled water was added thereto to obtain 170 g of a siloxane solution with a basic functional group (N2) whose solid content was approximately 15.1% in concentration.

### Synthesis Example 3

Forty grams of an organic silica compound (262.32→193.32) represented by the following formula:

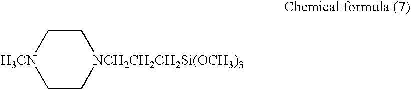

was added dropwise to 200 g of distilled water and hydrolyzed at room temperature for 3 days.

The resultant oligomer solution was concentrated at 60° C. with an evaporator. Thereafter, 70 g of distilled water was added thereto to obtain approximately 200 g of a siloxane solution with a basic functional group (N3) whose solid content was 14.7% in concentration.

### Synthesis Example 4

Five parts by weight of double strand DNA (molecular weight: 6×10⁶) obtained from a salmon soft roe (testis) was dissolved in 1000 parts by weight of ion-exchanged water over one day to obtain a aqueous DNA solution.

### Example 1

To 100 parts by weight of 30% (w/w) silica sol (SNOWTEX CM, NISSAN CHEMICAL INDUSTRIES, LTD.), 11 parts by weight of the siloxane solution (N1) obtained in Synthesis example 1 was added and slowly stirred for 10 minutes. Then, 160 parts by weight of the aqueous DNA solution obtained in Synthesis example 4 was added thereto. Using an evaporator, a dispersion solvent was removed at 50° C. with slow stirring. The resultant solution was dried at 60° C. for 15 hours to obtain a DNA carrier 1 having a DNA content of approximately 2.5% (w/w).

This DNA carrier 1 was subjected to an elution test. To 50 parts by weight of ion-exchanged water, 0.1 parts by weight of a bulk of the DNA carrier 1 was added. The mixture was left undisturbed at room temperature for 48 hours under closed conditions. The absorbance of DNA in the supernatant fluid measured at 260 nm using a spectrophotometer (U-3310, HITACHI) was 0.05. This result showed that 95% (w/w) of DNA was held in the DNA carrier.

The DNA carrier 1 was evaluated for the volume of a pore. After 0.5 parts by weight of the DNA carrier 1 was immersed in 10 parts by weight of ion-exchanged water for 5 hours, the DNA carrier 1 was transferred to a nylon mesh, and adsorption water on its surface was instantly splashed with an air gun. When the weight of the resultant water-soaked DNA carrier 1 was measured, the weight grew 16% to 0.58 parts by weight.

In 5 parts by weight of an ethidium bromide aqueous solution of 50 ppm, 0.5 parts by weight of the DNA carrier 1 was immersed. After 3 hours into the immersion, coloring by ethidium bromide in the supernatant decreased and the DNA carrier 1 turned red. The DNA carrier 1 showed orange fluorescence by ultraviolet irradiation at 360 nm. This demonstrated that the intercalation capability for the harmful compound having a planer structure was maintained.

In addition, the specific surface of this DNA carrier 1 measured by a nitrogen adsorption method was 135 m²/g.

### Example 2

To 100 parts by weight of 30% (w/w) silica sol (SNOWTEX CM, NISSAN CHEMICAL INDUSTRIES, LTD.), 5 parts by weight of the siloxane solution (N2) obtained in Synthesis example 2 was added and slowly stirred for 10 minutes. Then, 160 parts by weight of the aqueous DNA solution obtained in Synthesis example 4 was added thereto. Using an evaporator, a dispersion solvent was removed at 50° C. with slow stirring. The resultant solution was dried at 60° C. for 15 hours to obtain a DNA carrier 2 having a DNA content of approximately 2.5% (w/w).

When the DNA carrier 2 was evaluated for increase in weight in ion-exchanged water in the same way as Example 1, the weight grew 18% for 5 hours.

This DNA carrier 2 was subjected to an elution test in the same way as Example 1. The absorbance of DNA in the supernatant was approximately 0.03. This result showed that 97% (w/w) of DNA was held in the DNA carrier.

### Example 3

To 100 parts by weight of 30% (w/w) silica sol (SNOWTEX CM, NISSAN CHEMICAL INDUSTRIES, LTD.), 15 parts by weight of the siloxane solution (N3) obtained in Synthesis example 3 was added and slowly stirred for 10 minutes. Then, 250 parts by weight of the aqueous DNA solution obtained in Synthesis example 4 was added thereto. Using an evaporator, a dispersion solvent was removed at 50° C. with slow stirring. The resultant solution was dried at 60° C. for 15 hours to obtain a DNA carrier 3 having a DNA content of approximately 2.5% (w/w).

This DNA carrier 3 was subjected to an elution test in the same way as Example 1. The absorbance of DNA in the supernatant was approximately 0.05. This result showed that 95% (w/w) of DNA was held in the DNA carrier.

### Example 4

To 100 parts by weight of 30% (w/w) silica sol (SNOWTEX CM, NISSAN CHEMICAL INDUSTRIES, LTD.), 15 parts by weight of the siloxane solution (N3) obtained in Synthesis example 3 was added and slowly stirred for 10 minutes. Then, 160 parts by weight of the aqueous DNA solution obtained in Synthesis example 4 was added thereto. Using an evaporator, a dispersion solvent was removed at 50° C. with slow stirring. The resultant solution was dried at 60° C. for 15 hours to obtain a DNA carrier 4 having a DNA content of approximately 3.7% (w/w).

This DNA carrier 4 was subjected to an elution test in the same way as Example 1. The absorbance of DNA in the supernatant was approximately 0.05. This result showed that 95% (w/w) of DNA was held in the DNA carrier.

### Example 5

To 100 parts by weight of 30% (w/w) silica sol (SNOWTEX CM, NISSAN CHEMICAL INDUSTRIES, LTD.), 10 parts by weight of the siloxane solution (N2) obtained in Synthesis example 2 was added and slowly stirred for 10 minutes. Then, 100 parts by weight of the aqueous DNA solution obtained in Synthesis example 4 was added thereto. Using an evaporator, a dispersion solvent was removed at 50° C. with slow stirring. The resultant solution was dried at 60° C. for 15 hours to obtain a DNA carrier 5 having a DNA content of approximately 1.5% (w/w).

This DNA carrier 5 was subjected to an elution test in the same way as Example 1. The absorbance of DNA in the supernatant was approximately 0.01. This result showed that 98% (w/w) of DNA was held in the DNA carrier.

### Comparative Example 1

A silica powder having a specific surface of 250 m²/g was used to carry out a comparative test. To 5 parts by weight of the silica powder, 5 parts by weight of the DNA solution obtained in Synthesis example 4 was added and mixed to uniformly wet the silica powder. The resultant paste was dried at 50° C. for 24 hours to obtain a silica powder where the concentration of DNA held is 0.5% (w/w). The mixture of 0.1 parts by weight of the obtained silica powder and 50 parts by weight of ion-exchanged water was subjected to an elution test in the same way as Example 1. The absorbance of DNA in the supernatant was 0.16. This result showed that approximately 80% (w/w) of DNA was eluted.

### Comparative Example 2

To 100 parts by weight of 30% (w/w) silica sol (SNOWTEX CM, NISSAN CHEMICAL INDUSTRIES, LTD.), 11 parts by weight of the siloxane solution (N1) obtained in Synthesis example 1 was added and slowly stirred for 10 minutes. Using an evaporator, a dispersion solvent was then removed at 50° C. The resultant solution was dried at 60° C. for 15 hours to obtain siloxane-treated silica containing a basic functional group but no DNA. In 5 parts by weight of an ethidium bromide aqueous solution of 50 ppm, 0.5 parts by weight of the siloxane-treated silica containing a basic functional group but no DNA was immersed for 3 hours. However, coloring by ethidium bromide in the supernatant fluid hardly decreased. The siloxane-treated silica containing a basic functional group but no DNA showed no orange fluorescence even by ultraviolet irradiation at 360 nm.

As seen from the results, the DNA carrier of the present invention reduced the elution of DNA to water and efficiently collected a particular substance.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2004-207253 filed on Jul. 14, 2004, which is hereby incorporated by reference herein.

1. A DNA carrier, characterized in that DNA is held in a porous matrix containing polyorganosiloxane with a basic functional group and particles.

2. The DNA carrier according to claim 1, wherein the polyorganosiloxane with a basic functional group is polyorganosiloxane with an amino group.

3. The DNA carrier according to claim 2, wherein the polyorganosiloxane with an amino group is a hydrolysis condensate containing one or more of silane compounds represented by: 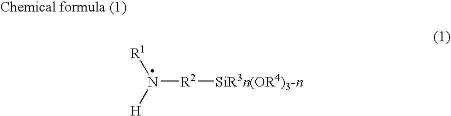
wherein R² represents a divalent carbohydrate group having 1 to 8 carbon atoms or a divalent group having —NH—; when R² represents a divalent carbohydrate group having 1 to 8 carbon atoms, R¹ represents a monovalent carbohydrate group having 1 to 8 carbon atoms, and when R² represents a divalent group having —NH—, R¹ represents H or a monovalent carbohydrate group having 1 to 8 carbon atoms; R³ and R⁴ each independently represent a monovalent carbohydrate group having 1 to 8 carbon atoms; and n represents 0, 1 or 2; 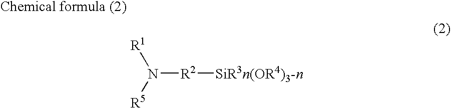
wherein R¹, R³, R⁴ and R⁵ each independently represent a monovalent carbohydrate group having 1 to 8 carbon atoms; R² represents a divalent carbohydrate group having 1 to 8 carbon atoms or a divalent group having —NH—; and n represents 0, 1 or 2; 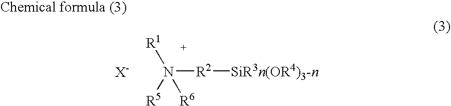
wherein R¹, R³, R⁴, R⁵ and R⁶ each independently represent a monovalent carbohydrate group having 1 to 8 carbon atoms; R² represents a divalent carbohydrate group having 1 to 8 carbon atoms or a divalent group having —NH—; n represents 0, 1 or 2; and X— represents an anion; 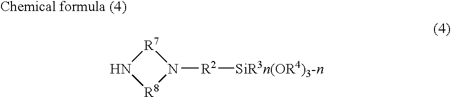
wherein R³ and R⁴ each independently represent a monovalent carbohydrate group having 1 to 8 carbon atoms; R⁷ and R⁸ each independently represent a divalent carbohydrate group; R² represents a divalent carbohydrate group having 1 to 8 carbon atoms or a divalent group having —NH—; and n represents 0, 1 or 2; 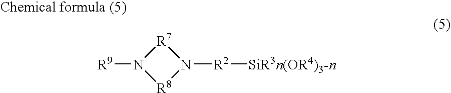
(wherein R³, R⁴ and R⁹ each independently represent a monovalent carbohydrate group having 1 to 8 carbon atoms; R⁷ and R⁸ each independently represent a divalent carbohydrate group; R² represents a divalent carbohydrate group having 1 to 8 carbon atoms or a divalent group having —NH—; and n represents 0, 1 or 2.

4. The DNA carrier according to claim 1, wherein the particles each have a particle size of 5 to 100 nm.

5. The DNA carrier according to claim 4, wherein the particles contain an oxide.

6. The DNA carrier according to claim 5, wherein the oxide contains colloidal silicon dioxide.

7. The DNA carrier according to claim 1, wherein the DNA carrier has a DNA content of 0.01 to 15% (w/w).

8. A method of producing a DNA carrier according to any one of claims 1 to 7, comprising the steps of: preparing a dispersion/dissolution solution in which particles, DNA and polyorganosiloxane with a basic functional group are dispersed and dissolved; and removing a dispersion solvent from the dispersion/dissolution solution.

9. A collection system using DNA, comprising means for bringing water and/or gas containing a substance that can be collected by DNA into contact with a DNA carrier according to any one of claims 1 to 7.

10. The collection system using DNA according to claim 9, wherein the system is used in an environmental cleanup system.
